(19) United States
(12) Patent Application Publication
(10) Pub. No.: US 2026/0021313 A1
(43) Pub. Date: Jan. 22, 2026

(54) DISTAL ELECTRODE FOR IMPLANTABLE MEDICAL DEVICE

(71) Applicant: Medtronic, Inc., Minneapolis, MN (US)

(72) Inventors: Thomas A. Anderson, New Hope, MN (US); Zhongping Yang, Woodbury, MN (US); Jason D. Hamack, Ramsey, MN (US)

(21) Appl. No.: 19/271,877

(22) Filed: Jul. 17, 2025

Related U.S. Application Data
(60) Provisional application No. 63/672,487, filed on Jul. 17, 2024.

(51) Int. Cl. A61N 1/375; A61N 1/05

(52) U.S. Cl. CPC A61N 1/3756; A61N 1/0573; A61N 1/37512

(57) ABSTRACT

A device comprising: an elongated housing extending from a proximal end to a distal end along a longitudinal axis, the elongated housing being configured to be implanted wholly within a chamber of a heart; an elongated body extending distally from the distal end of the elongated housing, the elongated body comprising: a proximal portion extending distally from the distal end of the elongated housing, a medial portion coupled to and distal to the proximal portion, the medial portion defining a first electrode, and
a distal portion coupled to and distal to the medial portion, the distal portion defining a distal end of the elongated body, wherein the distal portion is configured to penetrate wall tissue of the chamber; and a second electrode disposed on the distal end the elongated housing, the second electrode being configured to interface with the wall tissue of the chamber without penetrating the wall tissue.

### RELATED APPLICATIONS

This application claims the benefit of U.S. Provisional Application Ser. No. 63/672,487, filed Jul. 17, 2024, the entire contents of which are incorporated herein by reference.

### TECHNICAL FIELD

The disclosure relates to medical devices, and more particularly to fixation mechanisms of medical devices.

### BACKGROUND

Various types of implantable medical devices (IMDs) have been implanted for treating or monitoring one or more conditions of a patient. Such IMDs may be adapted to monitor or treat conditions or functions relating to heart, muscle, nerve, brain, stomach, endocrine organs or other organs and their related functions. Such IMDs may be associated with leads that position electrodes at a desired location or may be leadless with electrodes integrated with and/or attached to the device housing. These IMDs may have the ability to wirelessly transmit data either to another device implanted in the patient or to another instrument located externally of the patient, or both.

A cardiac pacemaker is an IMD configured to deliver cardiac pacing therapy to restore a more normal heart rhythm. Such IMDs sense the electrical activity of the heart, and deliver cardiac pacing based on the sensed electrical activity, via electrodes. Some cardiac pacemakers are implanted a distance from the heart and coupled to one or more leads that intravascularly extend into the heart to position electrodes with respect to cardiac tissue. Some cardiac pacemakers are sized to be completely implanted within one of the chambers of the heart and may include electrodes integrated with or attached to the device housing rather than leads. Some cardiac pacemakers provide dual chamber functionality, by sensing and/or stimulating the activity of both atria and ventricles, or other multi-chamber functionality. A cardiac pacemaker may provide multi-chamber functionality via leads that extend to respective heart chambers, or multiple cardiac pacemakers may provide multi-chamber functionality by being implanted in respective chambers.

### SUMMARY

In general, this disclosure is directed to implantable medical devices (IMDs) configured to sense and deliver electrical signals to tissue of a patient via a plurality of electrodes at or near a distal end of an elongated housing of the IMD. More particularly, this disclosure is directed to IMDs with a electrode disposed at or near the distal end of the elongated housing.

In some examples, a single IMD is implanted in one chamber of a heart of the patient and is able to sense electrical activity in and/or deliver cardiac pacing to more than one chamber, which may avoid the need for a leaded device or multiple smaller devices to provide such functionality, which may reduce the amount of material implanted within the patient. In some examples, such an implantable medical device includes a distal electrode that is configured to penetrate through wall tissue of the heart chamber in which the device is implanted, and into wall tissue of another heart chamber. In addition to the distal electrode, the device includes a reference electrode and one or more proximal electrodes configured to contact the wall tissue of the heart chamber. The distal electrode may be a helix configured to penetrate tissue of the patient. The distal electrode may be configured to sense in and/or deliver cardiac pacing to one chamber of the heart and the one or more proximal electrodes may be configured to sense in and/or deliver cardiac pacing to another separate chamber of the heart.

The distal electrode may be a part of an elongated body extending distally from a distal end of the IMD. The distal electrode may be an electrically conductive region proximal to a distal end of the elongated body. Placement of the electrically conductive region proximal to the distal end may reduce the effect of any lateral movement of the distal end on delivery of cardiac pacing by the first electrode and/or reduce pacing capture thresholds of cardiac tissue by positioning the first electrode proximate to cardiac tissue with reduced inflammation and/or irritation.

In some examples, this disclosure is directed to a device comprising: an elongated housing extending from a proximal end to a distal end along a longitudinal axis, the elongated housing being configured to be implanted wholly within a chamber of a heart; an elongated body extending distally from the distal end of the elongated housing, the elongated body comprising: a proximal portion extending distally from the distal end of the elongated housing, a medial portion coupled to and distal to the proximal portion, the medial portion defining a first electrode, and a distal portion coupled to and distal to the medial portion, the distal portion defining a distal end of the elongated body, wherein the distal portion is configured to penetrate wall tissue of the chamber; and a second electrode disposed on the distal end of the elongated housing, the second electrode being configured to interface with the wall tissue of the chamber without penetrating the wall tissue.

In some examples, this disclosure is directed to a method comprising: penetrating, via an elongated body of an implantable medical device (IMD), tissue within a first chamber of a heart of a patient, wherein the elongated body comprises: a proximal portion extending distally from a distal end of an elongated housing of the IMD along a longitudinal axis of the IMD, a medial portion coupled to and distal to the proximal portion, the medial portion defining a first electrode, and a distal portion coupled to and distal to the medial portion, the distal portion defining a distal end of the elongated body; advancing the elongated body of the IMD into the tissue of the heart until a second electrode of the IMD contacts the wall tissue without penetrating the tissue, wherein when the second electrode contacts the tissue, the distal end of the elongated body is positioned at a first location within the tissue of the heart; delivering, via the first electrode of the IMD, cardiac pacing to a second chamber of the heart, wherein the second chamber is separate from the first chamber, wherein delivering the cardiac pacing to the second chamber comprises: delivering, via the first electrode, the cardiac pacing to a second location within the tissue of the heart, the second location being proximal to the first location along the longitudinal axis; and delivering, via the second electrode of the IMD, cardiac pacing to the first chamber of the heart.

In some examples, this disclosure is directed to a method comprising: disposing, by a manufacturing process, an electrically insulating material over an outer surface of an elongated body, the elongated body comprising: a proximal portion; a medial portion coupled to and distal to the proximal portion; and a distal portion coupled to and distal to the medial portion, the distal portion defining a distal end of the elongated body; removing, by the manufacturing process, a portion of the electrically insulating material around the medial portion of the elongated body to expose an outer surface of the medial portion; and either before or after said disposing, assembling, by the manufacturing process, the elongated body to an implantable medical device (IMD), the IMD comprising: an elongated housing extending from a proximal end to a distal end along a longitudinal axis, the elongated housing being configured to be implanted wholly within a chamber of a heart.

In some examples, this disclosure is directed to a medical device comprising: a housing extending from a proximal end to a distal end along a longitudinal axis, the housing being configured to be implanted wholly within a chamber of a heart; a helix extending in a distal winding direction from the distal end of the elongated housing, the helix comprising a distal portion configured to penetrate wall tissue of the chamber; a first electrode formed or carried by the helix, the first electrode being located at a position on the helix that is spaced in a proximal winding direction from the distal portion of the helix.

This summary is intended to provide an overview of the subject matter described in this disclosure. It is not intended to provide an exclusive or exhaustive explanation of the methods and systems described in detail within the accompanying drawings and description below.

### BRIEF DESCRIPTION OF DRAWINGS

The details of one or more examples of this disclosure are set forth in the accompanying drawings and the description below. Other features, objects, and advantages of this disclosure will be apparent from the description and drawings, and from the claims.

FIG. 1 is a conceptual diagram illustrating an example device implanted in the heart of a patient, in accordance with one or more aspects of this disclosure.

FIG. 2 is a perspective diagram illustrating an example configuration of the device of FIG. 1.

FIG. 3 is a perspective diagram illustrating a top view of the example device of FIG. 2.

FIG. 4 is a perspective diagram illustrating a side view of the example device of FIG. 2.

FIG. 5 is a block diagram illustrating an example configuration of an example device of any of FIGS. 1-4.

FIG. 6 is a conceptual diagram of an example device of any of FIGS. 1-5 implanted at one example of a target implant site.

FIG. 7 is a flowchart illustrating an example process for sensing a cardiac electrical signal and delivering cardiac pacing therapy to a heart of a patient via an example device of any of FIGS. 1-6.

### DETAILED DESCRIPTION

In general, this disclosure is directed to distal end configurations for implantable medical devices (IMDs). More particularly, this disclosure is directed to IMDs with an elongated body extending distally from the distal end of the IMD. The elongated body may define an electrode proximal to a distal end of the elongated body. Placement of the electrode proximal to the distal end of the elongated body may reduce the relative movement of the electrode within tissue, allow for a use of a larger elongated body on the IMD, reduce tissue irritation around the electrode, and/or reduce the need to use therapeutic substances during implantation of the IMD.

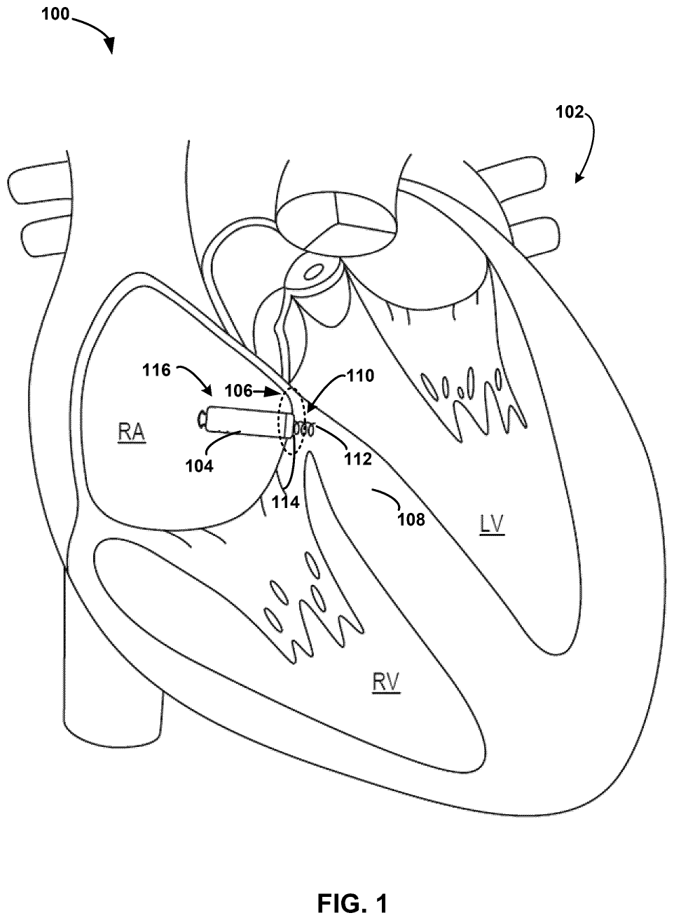

FIG. 1 is a conceptual diagram illustrating an example device 104 implanted in the heart 102 of a patient, in accordance with one or more aspects of this disclosure. Device 104 is shown implanted in the right atrium (RA) of the patient's heart 102 in a target implant region 106, such as the triangle of Koch, in heart 102 of the patient with a distal end of device 104 directed toward the left ventricle (LV) of the patient's heart 102. Although in the example of FIG. 1 the distal end of device 104 is directed toward the LV, the distal end may be directed to other targets, such as interventricular septum of heart 102. Target implant region 106 may lie between the bundle of His and the coronary sinus and may be adjacent the tricuspid valve. In some examples, target implant region 106 may be disposed in another position within heart 102, e.g., within a right ventricle (RV) of heart 102.

Device 104 includes a distal end 110 and a proximal end 116. Distal end 110 includes a first electrode 112, and a second electrode 114. First electrode 112 may be disposed along an elongated body extending to a distal end. In such examples, first electrode 112 may be disposed along the elongated body at a position proximal to the distal end of the elongated body. The elongated body may define a helical shape, an elongated barb, or any other elongated shape. First electrode 112 may define the entire elongated body and an electrically active region of first electrode 112 may be disposed proximal of the distal end of the elongated body.

The elongated body and first electrode 112 extends from distal end 110 and may penetrate into and/or through the wall tissue of a first chamber (e.g., the RA in the illustrated example) into wall tissue of a second chamber (e.g., ventricular myocardium 108 of the LV in the illustrated example). The distal end of the elongated body may experience increased movement within the wall tissue relative to a more proximal portion of the elongated body. In some examples, the wall tissue around the distal end of the elongated body may be more inflamed and/or irritated than other wall tissue surrounding the elongated body, e.g., due to the penetration of the distal end of the elongated body into the wall tissue. Placement of first electrode 112 and/or an electrically active region of first electrode 112 proximal to the distal end reduces movement of first electrode 112 and/or the electrically active region within the wall tissue, e.g., which may allow for more efficacious delivery of cardiac pacing signals to heart 102. In some examples, placement of first electrode 112 and/or the electrically active region proximal to the distal end allows for delivery of cardiac pacing signals to portions of the wall tissue that are relatively less inflamed and/or irritated, e.g., which may reduce pacing capture thresholds for the wall tissue and allow for more efficacious delivery of cardiac pacing signals to heart 102.

Second electrode 114 may be a compliant electrode. Second electrode 114 may include, but is not limited to, a wave spring electrode, a coil spring electrode, a button electrode, or the like. Second electrode 114 may contact the wall tissue of the first chamber as first electrode 112 penetrates the wall tissue of the first chamber. When device 104 is affixed to the wall tissue of the first chamber, second electrode 114 may place an electrically active region of second electrode 114 in contact with the wall tissue (e.g., without puncturing or penetrating the wall tissue).

The configuration of electrodes 112 and 114 illustrated in FIG. 1 allows device 104 to sense cardiac signals and/or deliver cardiac pacing to multiple chambers of heart 102, e.g., the RA and ventricle(s) in the illustrated example. In this manner, the configuration of electrodes 112 and 114 may facilitate the delivery of multi-chamber pacing (e.g. DDD pacing) and/or A-V synchronous pacing by single device 104 implanted within the single chamber, e.g., the RA. While device 104 is implanted at target implant region 106 to sense and/or pace the RA and ventricle(s) in the example shown in FIG. 1, a device having an electrode configuration in accordance with the examples of this disclosure may be implanted at any of a variety of locations to sense in and/or pace any one, two or more chambers of heart 102. For example, device 104 may be implanted at region 106 or another region, and first electrode 112 may extend into tissue, e.g., myocardial tissue, of the LV or interventricular septum to, for example, facilitate the delivery of A-V synchronous pacing. Furthermore, a device having an electrode configuration in accordance with the examples of this disclosure may be implanted at any of a variety of locations within a patient for sensing and/or delivery of therapy to other patient tissue. Such locations include any of the chambers of the heart, in the right atrium at the triangle of Koch or elsewhere therein, in the right ventricle at or near the apex thereof, or in the interventricular septum in the right ventricle, e.g. with the device implanted in the septum so as to enable conduction system pacing (CSP) such as left bundle branch area pacing (LBBAP) with first electrode 112 selectively or non-selectively capturing the left bundle branch (LBB); in such a CSP/LBBAP application (or otherwise), electrode 114 may optionally be omitted. In some examples, first electrode 112 extends into the tissue of heart 102 at region 106 and affixes device 104 to the tissue of heart 102.

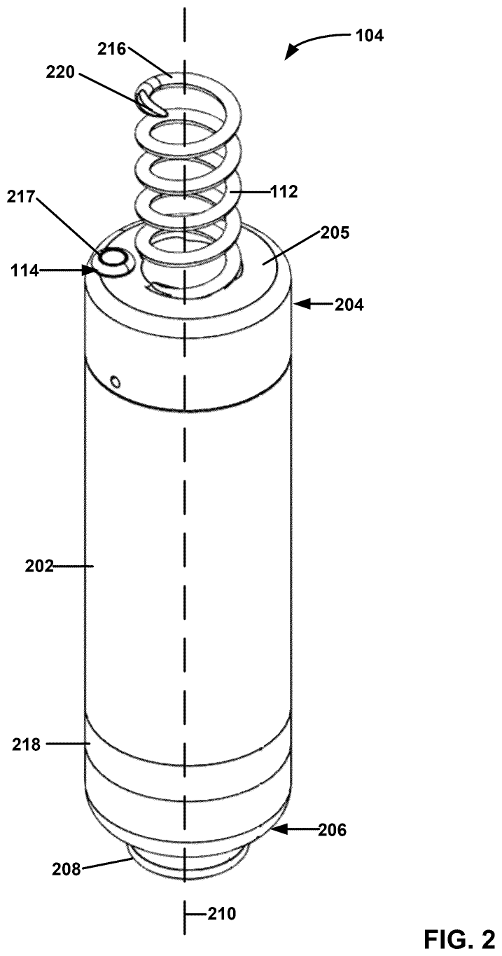

FIG. 2 is a perspective diagram illustrating an example configuration of the device of FIG. 1. Device 104 may include a housing 202 extending from a distal end 204 to a proximal end 206 along longitudinal axis 210. First electrode 112 and second electrode 114 may extend distally from distal end 204 of housing 202 and along longitudinal axis 210. While FIG. 2 illustrates first electrode 112 as an elongated helix, other example devices 104 may include first electrode 112 defining an elongated barb or other elongated body.

Housing 202 may define a hermetically sealed internal cavity. Housing 202 may be formed from a conductive material including titanium or titanium alloy, stainless steel, MP35N (a non-magnetic nickel-cobalt-chromium-molybdenum alloy), platinum alloy or other bio-compatible metal or metal alloy, or other suitable conductive material. In some examples, housing 202 is formed from a non-conductive material including ceramic, glass, sapphire, silicone, polyurethane, epoxy, acetyl co-polymer plastics, polyether ether ketone (PEEK), a liquid crystal polymer, other biocompatible polymer, or other suitable non-conductive material.

Housing 202 extends between distal end 204 and proximal end 206 along longitudinal axis 210. Housing 202 may be cylindrical or substantially cylindrical but may be other shapes, e.g., prismatic, or other geometric shapes. Housing 202 may include a delivery tool interface member 208, e.g., at proximal end 206, for engaging with a delivery tool during implantation of device 104. At distal end 204, housing 202 may define a face 205 of housing 202. Face 205 may define a distal end major surface. Face 205 may be orthogonal to longitudinal axis 210. In some examples, face 205 is slanted, e.g., face 205 defines a reference plane that is not orthogonal to longitudinal axis 210.

Face 205 may define a distal end of housing 202. Electrodes 112 and 114 may extend distally from face 205 along longitudinal axis 210. In some examples, device 104 includes one or more fixation features (e.g., recesses, protrusions, ramps, meshes, tines, or the like) disposed on face 205. First electrode 112 may define a helical or spiral structure. First electrode 112 may extend distally from face 205 to a distal tip. Second electrode 114 may be disposed on face 205. Second electrode 114 may be disposed radially outwards of first electrode 112 and radially inwards of an outer perimeter of face 205. Second electrode 114 may include, but is not limited to, a button electrode, a ring electrode, or a spring electrode. A distal surface (e.g., a second electrically active region 217) of second electrode 114 may be proud of (e.g., longitudinally distal of) face 205 or may be flush with face 205.

First electrode 112 may include one or more coatings (e.g., electrically insulative coating(s)) configured to define a first electrically active region 216, or first electrode 112 may otherwise define first electrically active region 216. First electrically active region 216 may be proximal to distal end 220 of first electrode 112. First electrically active region 216 may be longitudinally and/or circumferentially offset from distal end 220, e.g., by at least a threshold distance. The separation between first electrically active region 216 and distal end 220 may reduce movement of first electrically active region 216 within tissue once device 104 is implanted. The separation between first electrically active region 216 and distal end 220 may position first electrically active region 216 around tissue with reduced inflammation and/or irritation when device 104 is implanted within tissue, e.g., thereby reducing pacing capture thresholds for cardiac pacing signals delivered by device 104 through first electrically active region 216. Second electrode 114 may include one or more coatings configured to define a second electrically active region 217 on an outer surface of second electrode 114.

First and second electrodes 112 and 114 may be formed of an electrically conductive material, such as titanium, platinum, iridium, tantalum, stainless steel or alloys thereof. In some examples, second electrode 114 is formed from one or more of Platinum Iridium, a Platinum Iridium-clad alloy (e.g., Platinum Iridium-clad Titanium or Nitinol), Nitinol, or Tantalum Tungsten. First and second electrodes 112 and 114 may be coated with an electrically insulating coating, e.g., a parylene, polyurethane, silicone, epoxy, or other insulating coating, to reduce the electrically conductive active surface area of first and second electrodes 112 and 114, and thereby define first and second electrically active regions 216 and 217. Defining first and second electrically active regions 216 and 217 by covering portions with an insulating coating may increase the electrical impedance of first and second electrodes 112 and 114 and thereby reduce the current delivered during a pacing pulse that captures the cardiac tissue. A lower current drain conserves the power source, e.g., one or more rechargeable or non-rechargeable batteries, of device 104.

In some examples, first and second electrodes 112 and 114 include an electrically conducting material coating on first and second electrically active regions 216 and 217 to define the active regions. For example, first and second electrically active regions 216 and 217 may be coated with titanium nitride (TiN). First and second electrodes 112 and 114 may be made of substantially similar material or may be made of different material from one another.

In the example of FIG. 2, first electrode 112 takes the form of a helix or a coil. First electrode 112 may be an elongated body defining a helix. In some examples, a helix is an object having a three-dimensional shape like that of a wire wound uniformly in a single layer around a cylindrical or conical surface or mandrel such that the wire would be in a straight line if the surface were unrolled into a plane. First electrode 112 may extend from face 205 from a proximal end to distal end 220, e.g., defining first electrically active region 216 proximal to distal end 220. The proximal end may be a location along first electrode 112 where first electrode 112 extends distally past face 205 of device 104. In some examples, first electrode 112 takes the form of an elongated barb or other elongated body configured to penetrate into tissue.

In some examples, first electrode 112 includes one or more anti-rotation features. The anti-rotation features may facilitate fixation of first electrode 112 to the tissue. The additional anti-rotation features may include a shape of first electrode 112, dimensions (e.g., outer diameter, pitch, or the like) of first electrode 112, one or more features disposed on an outer surface of first electrode 112, or the like. The shape and/or dimensions of first electrode 112 may include a geometric shape of first electrode 112, a varying diameter configuration of first electrode 112, a varying pitch configuration of first electrode 112, a waveform configuration of first electrode 112, or any combination herein. The one or more anti-rotation features disposed on first electrode 112 may include, but are not limited to, elongated darts, barbs, or tines. In some examples, the anti-rotation features include bumps, ridges, recesses, and/or other texturing disposed on face 205. The one or more anti-rotation features may resist rotation of first electrode 112, e.g., by penetrating the tissue, by increasing the friction between first electrode 112 and the tissue, or the like.

First and second electrodes 112 and 114 may vary in size and shape in order to enhance tissue contact of first and second electrically active regions 216 and 217. For example, first electrodes 112 may have a round cross-section or could be made with a flatter cross-section (e.g., oval or rectangular) based on tissue contact specifications. In some examples, second electrode 114 defines an outer surface that varies in size and shape (e.g., an oval outer surface, an outer surface with a larger diameter, or the like) in order to enhance tissue contact of second electrically active region 217.

Distal end 220 of first electrode 112 can have a conical, hemi-spherical, or slanted edge distal tip with a narrow tip diameter, e.g., less than 1 millimeter (mm), for penetrating into and through tissue layers. Distal end 220 can be a sharpened or angular tip or sharpened or beveled edges, but the degree of sharpness may be constrained to avoid a cutting action that could lead to lateral displacement of distal end 220 of first electrode 112 and undesired tissue trauma. Distal end 220 is electrically insulated and first electrically active region 216 is longitudinally proximal to distal end 220, e.g., to reduce an effect of lateral displacement of distal end 220 within the tissue on the delivery of cardiac pacing signals by device 104 to the tissue via first electrically active region 216. For example, reduced lateral displacement of first electrically active region 216 within the tissue may increase the efficacy of the cardiac pacing signals, e.g., by increasing the consistency of the delivery of cardiac pacing signals to tissue within target implant region 106. In some examples, first electrode 112 defines a maximum diameter at its base that interfaces with housing distal end 204. In such examples, the outer diameter of the helix defined by first electrode 112 may decrease from housing distal end 204 to the distal end of first electrode 112. In some examples, the diameter of first electrode 112 varies from a proximal end to the distal end of first electrode 112. The varying diameter may cause first electrode 112 to resist rotation within the tissue of heart 102.

Placement of first electrically active region 216 proximal to distal end 220 may allow for first electrode 112 with an increased outer diameter (e.g., an increased cross-sectional diameter) than an otherwise identical first electrode 112 with first electrically active region 216 at or around distal end 220. First electrode 112 with a larger outer diameter may lead to a relatively stiffer helix, which may increase fixation of device 104 within the tissue and/or lead to increased lateral movement of distal end 220 of first electrode 112. Placement of first electrically active region 216 proximal to distal end 220 may reduce the effects of the lateral movement of distal end 220, e.g., on pacing thresholds, and/or reduce damage to the tissue by first electrode 112.

The outer dimensions of first electrode 112 can be substantially straight and cylindrical, with first electrode 112 being rigid in some examples. First electrode 112 may have flexibility in lateral directions, being non-rigid to allow some flexing with heart motion. In a relaxed state, when not subjected to any external forces, first electrode 112 can be configured to maintain a distance between first electrically active region 216 and housing distal end 204 and/or between first electrically active region 216 and distal end 220.

Distal end 220 of first electrode 112 can pierce through one or more tissue layers to position first electrically active region 216 within a desired tissue layer, e.g., the ventricular myocardium 108 or interventricular septum, at a position proximal to distal end 220. Accordingly, first electrode 112 extends a distance from housing distal end 204 corresponding to the expected pacing site depth and may have a relatively high compressive strength along its longitudinal axis, which may be substantially similar to or coincident with longitudinal axis 210, to resist bending in a lateral or radial direction when a longitudinal, axial, and/or rotational force is applied, e.g., to the proximal end 206 of housing 202 to advance device 104 into the tissue at target implant region 106. The expected pacing site depth may be less than the implantation depth of distal end 220. By resisting bending in a lateral or radial direction, first electrode 112 can maintain a spacing between a plurality of windings of first electrode 112 when first electrode 112 is a helix electrode. The spacing may be a pre-determined pitch of first electrode 112 and may vary from distal end 204 to distal end 220 of first electrode 112. First electrode 112 may be longitudinally non-compressible. First electrode 112 may also be elastically deformable in lateral or radial directions when subjected to lateral or radial forces, however, to allow temporary flexing, e.g., with tissue motion, but returns to its normally straight position when lateral forces diminish. In some examples, when first electrode 112 is not exposed to any external force, or to only a force along its longitudinal axis (substantially similar to or coincident with longitudinal axis 210), first electrode 112 retains a straight, linear configuration as shown.

As first electrode 112 enters tissue, second electrode 114 may at least partially contact a surface of the tissue. Second electrode 114 may interface with the surface of the tissue without penetrating the surface of the tissue. Second electrode 114 may sense signals from and/or deliver cardiac pacing signals to the tissue surface. As first electrode 112, at least a portion of one or more fixation features disposed on face 205 may be placed into contact with the tissue surface and may interface with the tissue surface to inhibit unintended movement of device 104 relative to the tissue surface.

All, substantially all, or a portion of housing 202 may function as an electrode 218, e.g., an anode, during pacing and/or sensing. In some examples, electrode 218 circumscribes a portion of housing 202 at or near proximal end 206. Electrode 218 can fully or partially circumscribe housing 202. FIG. 2 shows electrode 218 extending as a singular band around the outer perimeter of housing 202. Electrode 218 can also include multiple segments spaced a distance apart along a longitudinal axis 210 of housing 202 and/or around a perimeter of housing 202.

When housing 202 is formed from a conductive material, such as a titanium alloy, portions of housing 202 may be electrically insulated by a non-conductive material, such as a coating of parylene, polyurethane, silicone, epoxy or other biocompatible polymer, or other suitable material. For the portions of housing 202 without the non-conductive material, one or more discrete areas of housing 202 with conductive material can be exposed to define electrode 218.

When housing 202 is formed from a non-conductive material, such as a ceramic, glass or polymer material, an electrically-conductive coating or layer, such as a titanium, platinum, stainless steel, alloys thereof, a conductive material may be applied to one or more discrete areas of housing 202 to form electrode 218.

In some examples, electrode 218 is a component, such as a ring electrode, that is mounted or assembled onto housing 202. Electrode 218 may be electrically coupled to internal circuitry of device 104 via electrically-conductive housing 202 or an electrical conductor when housing 202 is a non-conductive material. In some examples, electrode 218 is located proximate to proximal end 206 of housing 202 and can be referred to as a proximal housing-based electrode. Electrode 218 can also be located at other positions along housing 202, e.g., located proximately to distal end 204 or at other positions along longitudinal axis 210.

In some examples, second electrode 114 or electrode 218 is paired with first electrode 112 for sensing ventricular signals and delivering ventricular pacing pulses. In some examples, second electrode 114 is paired with electrode 218 or first electrode 112 for sensing atrial signals and delivering pacing pulses to atrial tissue (e.g., to the atrial endocardium) in target implant region 106. In other words, electrode 218 is paired, at different times, with first electrode 112 and/or second electrode 114 for either ventricular or atrial functionality, respectively. In some examples, first and second electrodes 112 and 114 are paired with each other, with different polarities, for atrial and ventricular functionality.

In some examples, second electrode 114 is configured as an atrial cathode electrode for delivering pacing pulses to the atrial tissue, e.g., at target implant region 106 in combination with electrode 218. Second electrode 114 and electrode 218 may also be used to sense atrial P-waves for use in controlling atrial pacing pulses (delivered in the absence of a sensed P-wave) and for controlling atrial-synchronized ventricular pacing pulses delivered using first electrode 112 as a cathode and electrode 218 as the return anode.

Distal end 220 and first electrically active region 216 of first electrode 112 can be configured to rest within a ventricular myocardium of the patient, and second electrode 114 can be configured to contact an atrial endocardium of the patient without penetration of the atrial endocardium. Device 104 may include more or fewer electrodes than two electrodes. In some examples, device 104 includes one or more second electrodes 114 along housing distal end 204. For example, device 104 may include two or three electrodes configured for atrial functionality like second electrode 114, and the three electrodes may be substantially similar or different from one another. Spacing between a plurality of second electrodes 114 may be at an equal or unequal distance. Second electrode(s) 114 may be individually selectively coupled to sensing and/or pacing circuitry enclosed by housing 202 for use as an anode with first electrode 112 or as an atrial cathode electrode, or may be electrically common and not individually selectable. In some examples, in place of first electrode 112, device 104 includes a fixation element (not shown) of similar shape and mechanical properties, but without an electrically active region or electrode formed thereon or borne thereby; in such examples, electrically active region 216 can be positioned on a separate member and/or on the housing 202. In some examples, device 104 only includes first electrode 112 and electrode 218 and does not include any second electrodes 114.

In some examples device 104 includes one or more therapeutic substance dispensing devices, e.g., on face 205. The therapeutic substance dispensing devices may be configured to elute one or more steroids to tissue in proximity to the therapeutic substance dispensing devices over time. The steroid may mitigate inflammation of patient tissue resulting from interaction with device 104. In some examples, the therapeutic substance dispensing devices comprises one or more monolithic controlled release devices (MCRDs).

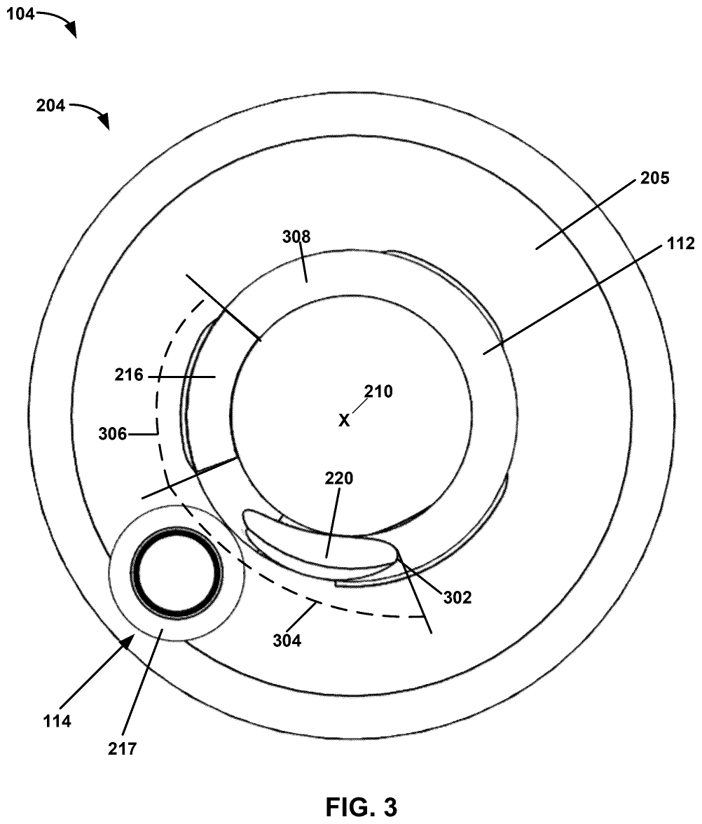

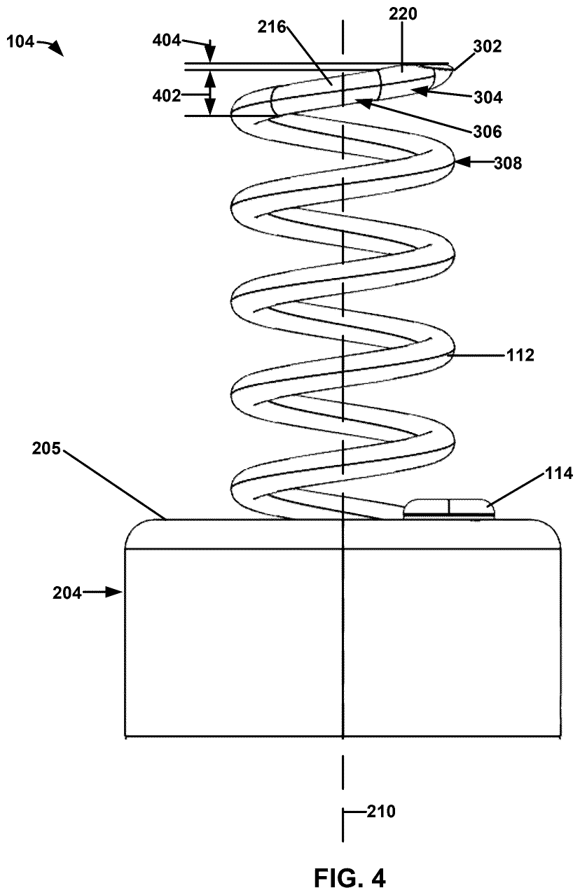

FIG. 3 is a perspective diagram illustrating a top view of device 104 of FIG. 2. FIG. 4 is a perspective diagram illustrating a side view of device 104 of FIG. 2. As illustrated in FIGS. 3 and 4, distal portion 204 of device 104 may include first electrode 112 extending distally from face 205 of device 104 along longitudinal axis 210 to distal end 220. Distal end 220 of first electrode 112 may terminate in a distal tip 302. First electrode 112 may define a proximal portion 308 extending from face 205, a medial portion 306 distal to and connected to proximal portion 308 and containing first electrically active region 216, and a distal portion 304 distal to and connected to medial portion 306 and containing distal end 220 of first electrode 112. Each of distal portion 304, medial portion 306, or proximal portion 308 may be formed from a same material or from different materials. In some examples, an electrically insulating coating is disposed over distal portion 304 and proximal portion 308, e.g., to define first electrically active region 216. In some examples, an electrically conductive material is disposed over medial portion 306, e.g., to define first electrically active region 216.

In some examples, as illustrated in FIG. 3, distal portion 304, medial portion 306, and proximal portion 308 are circumferentially from each other. In such examples, first electrically active region 216 may deliver cardiac pacing signals to tissue longitudinally and circumferentially offset from tissue at or around distal end 220 of first electrode 112. In some examples, distal portion 304 is longitudinally offset from and circumferentially overlapping with medial portion 306. In such examples, first electrically active region 216 may deliver cardiac pacing signals at a position longitudinally offset from and circumferentially aligned with distal end 220.

Longitudinal offset distance 404 between distal tip 302 and a distalmost edge of first electrically active region 216 may be at least 0.2 millimeters (mm). Longitudinal offset distance 404 allows for sufficient separation between distal end 220 and first electrically active region 216 to reduce the effects of any lateral movement of distal end 220 on first electrically active region 216 and/or to separate first electrically active region 216 from the more inflamed and/or irritated portions of tissue around first electrode 112. The circumferential offset between distal tip 302 and the distalmost edge of first electrically active region 216 may vary as a function of the pitch of the helix defined by first electrode 112, the cross-sectional diameter of first electrode 112, the outer diameter of first electrode 112, and/or longitudinal offset distance 404.

First electrically active region 216 may define a surface area of up to 5 square millimeters (mm²). In some examples, first electrically active region 216 defines a surface area of up to 3.5 mm². Longitudinal length 402 of first electrically active region 216 along longitudinal axis 210 may be a function of the pitch of the helix defined by first electrode 112, the cross-sectional diameter of first electrode 112, the outer diameter of first electrode 112, and/or the surface area of first electrically active region 216. In some examples, first electrically active region 216 defines longitudinal length 402 of up to 0.7 mm.

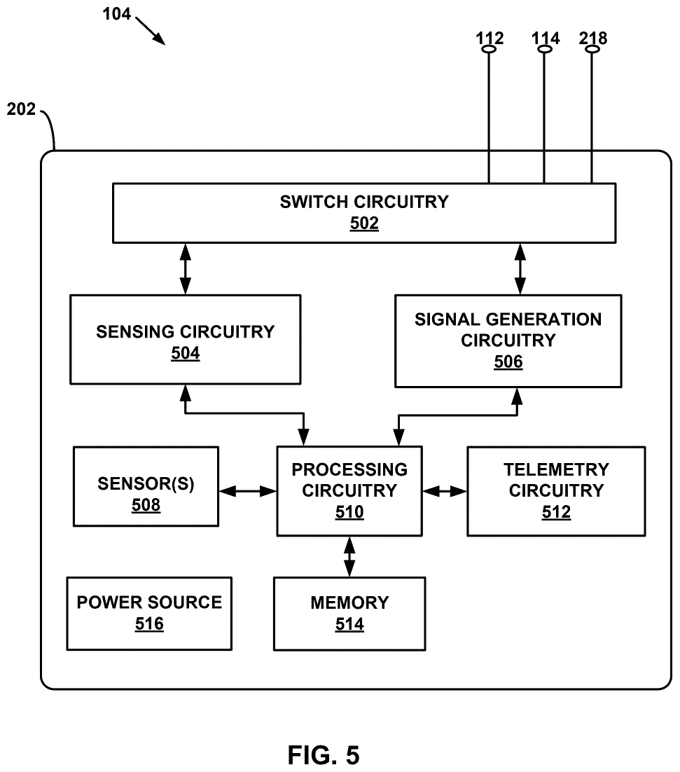

FIG. 5 is a block diagram illustrating an example configuration of an example device of any of FIGS. 1-4. In the example shown in FIG. 5, device 104 includes switch circuitry 502, sensing circuitry 504, signal generation circuitry 506, sensor(s) 508, processing circuitry 510, telemetry circuitry 512, memory 514, and power source 516. The various circuitry may be, or include, programmable or fixed function circuitry configured to perform the functions attributed to respective circuitry. Memory 514 may store computer-readable instructions that, when executed by processing circuitry 510, cause device 104 to perform various functions. Memory 514 may be a storage device or other non-transitory medium. The components of device 104 illustrated in FIG. 5 may be housed within housing 202.

Signal generation circuitry 506 generates electrical stimulation signals, e.g., cardiac pacing pulses. Switch circuitry 502 is coupled to electrodes 112, 114, and 218 and may include one or more switch arrays, one or more multiplexers, one or more switches (e.g., a switch matrix or other collection of switches), one or more transistors, or other electrical circuitry. Switch circuitry 502 is configured to direct stimulation signals from signal generation circuitry 506 to a selected combination of electrodes 112, 114, and 218, having selected polarities, e.g., to selectively deliver pacing pulses to the RA, ventricles, or interventricular septum of heart 102. For example, in order to pace one or both of the ventricles, switch circuitry 502 may couple first electrode 112, which has penetrated to wall tissue of a ventricle or the intraventricular septum, to signal generation circuitry 506 as a cathode, and one or both of second electrode 114 or electrode 218 to signal generation circuitry 506 as an anode. As another example, in order to pace the RA, switch circuitry 502 may couple second electrode 114, which maintains contact with the RA endocardium, to signal generation circuitry 506 as a cathode, and one or both of first electrode 112 or electrode 218 to signal generation circuitry 506 as an anode.

Each of electrodes 112, 114, 218 may be coupled to switch circuitry 502 via a corresponding feedthrough assembly. In some examples, each feedthrough assembly is substantially straight (e.g., along longitudinal axis 210). In some examples, such as when distal end 204 of housing 202 is removable from housing 202 (e.g., when distal end 204 is a removable header), the feedthrough assemblies are offset to allow for removal of distal end 204. For example, when a header defining distal end 204 is configured to be removably secured to housing 202 (e.g., via a turn-lock mechanism), the feedthrough assemblies are offset from longitudinal axis 210 to allow the header to turn relative to housing 202.

Switch circuitry 502 may also selectively couple sensing circuitry 504 to selected combinations of electrodes 112, 114, and 218, e.g., to selectively sense the electrical activity of either the RA or ventricles of heart 102. Sensing circuitry 504 may include filters, amplifiers, analog-to-digital converters, or other circuitry configured to sense cardiac electrical signals via electrodes 112, 114, and/or 218. For example, switch circuitry 502 may couple each of first electrode 112 and second electrode 114 (in combination with electrode 218) to respective sensing channels provided by sensing circuitry 504 to respectively sense either ventricular or atrial cardiac electrical signals. In some examples, sensing circuitry 504 is configured to detect events, e.g., depolarizations, within the cardiac electrical signals, and provide indications thereof to processing circuitry 510. In this manner, processing circuitry 510 may determine the timing of atrial and ventricular depolarizations, and control the delivery of cardiac pacing, e.g., AV synchronized cardiac pacing, based thereon. Processing circuitry 510 may include any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 510 herein may be embodied as firmware, hardware, software or any combination thereof.

Sensor(s) 508 may include one or more sensing elements that transduce patient physiological activity to an electrical signal to sense values of a respective patient parameter. Sensor(s) 508 may include one or more accelerometers, optical sensors, chemical sensors, temperature sensors, pressure sensors, or any other types of sensors. Sensor(s) 508 may output patient parameter values that may be used as feedback to control sensing and delivery of therapy by device 104.

Telemetry circuitry 512 supports wireless communication between device 104 and an external programmer (not shown in FIG. 5) or another computing device under the control of processing circuitry 510. Processing circuitry 510 of device 104 may receive, as updates to operational parameters from the computing device, and provide collected data, e.g., sensed heart activity or other patient parameters, via telemetry circuitry 512. Telemetry circuitry 512 may accomplish communication by radiofrequency (RF) communication techniques, e.g., via an antenna (not shown).

Power source 516 delivers operating power to various components of device 104. Power source 16 may include a rechargeable or non-rechargeable battery and a power generation circuit to produce the operating power. Recharging may be accomplished through proximal inductive interaction between an external charger and an inductive charging coil within device 104.

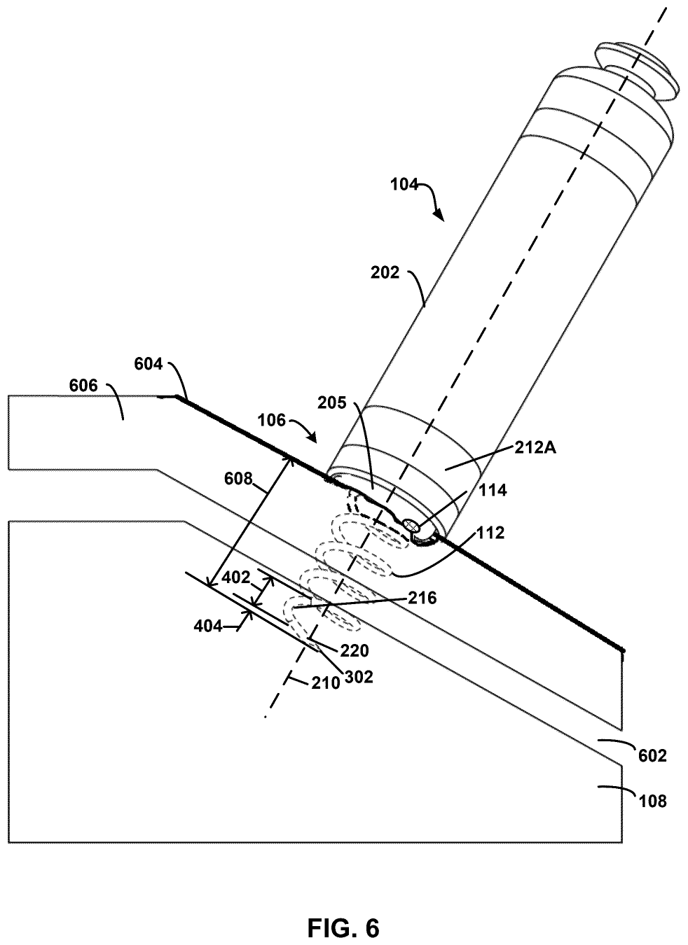

FIG. 6 is a conceptual diagram of device 104 of any of FIGS. 1-5 implanted at one example of a target implant location 106. First electrode 112 may be inserted (e.g., in a manner similar to rotating and advancing a threaded screw) such that tissue becomes engaged with the helix of first electrode 112. As first electrode 112 becomes engaged with tissue, distal end 220 of first electrode 112 (e.g., distal tip 302) pierces into the tissue at target implant region 106 and advances through atrial myocardium 606 and central fibrous body 602 to position first electrically active region 216 in ventricular myocardium 108 as shown in FIG. 6. In some examples, first electrode 112 penetrates into the interventricular septum. In some examples, first electrode 112 does not perforate either of the ventricular endocardial or epicardial surface.

In some examples, manual pressure applied to the housing proximal end 206, e.g., via an advancement tool, provides the longitudinal force to pierce the cardiac tissue at target implant region 106 (e.g., via distal tip 302 of first electrode 112). In some examples, actuation of an advancement tool rotates device 104 and first electrode 112 configured as a helix about longitudinal axis 210. The rotation of the helix about the longitudinal axis 210 advances distal end 220 of first electrode 112 through atrial myocardium 606 and central fibrous body 602 to position first electrically active region 216 in ventricular myocardium 108 as shown in FIG. 6.

As first electrode 112 advances into the tissue, the distance between second electrode 114 and atrial endocardium 604 decreases until second electrode 114 contact, and may press against, the surface of atrial endocardium 604. Second electrode 114 may press against atrial endocardium 604 without penetrating atrial endocardium 604.

Target implant region 106 in some pacing applications is along atrial endocardium 604, substantially inferior to the AV node and bundle of His. For example, target implant region 106 may be at least partially within the Triangle of Koch of heart 102. First electrode 112 can have a length that penetrates through atrial 604 in target implant region 106, through the central fibrous body 602 and into ventricular myocardium 108 without perforating through the ventricular endocardial surface. In some examples, when the full length of first electrode 112 is fully advanced into target implant region 106, first electrically active region 216 rests within ventricular myocardium 108 and second electrode 114 is positioned in intimate contact with atrial endocardium 604. First electrode 112 may extend from housing distal end 204 approximately 3 mm to 12 mm in various examples. In some examples, first electrode 112 may extend a distance from distal end 204 by at least 3 mm. The diameter of an elongated body defining first electrode 112 may be 4 mm or less, e.g., may be 1 mm or less, may be 0.6 mm or less. An outer diameter of the helix or coil defined by first electrode 112 may be up to 6 mm (e.g., up to 4 mm).

Once face 205 and/or second electrode 114 is in contact with atrial endocardium 604, distal end 220 of first electrode 112 may within cardiac tissue of heart 102 at up to distance 608 from the surface of atrial endocardium 604 along longitudinal axis 210. Distance 608 may be up to 10 mm (e.g., up to 6 mm). Distance 608 may be measured from distal tip 302 of distal end 220 of first electrode 112. When distal tip 302 is at distance 608 from the surface of atrial endocardium 604, first electrically active region 216 may be separated from distal tip 302 by at least longitudinal offset distance 404 along longitudinal axis 210. First electrically active region 216 may encompass longitudinal length 402 along longitudinal axis 210. When device 104 is implanted into ventricular myocardium 108, device 104 may be configured to deliver cardiac pacing signals to ventricular myocardium 108 at a position proximal to distal end 302 by at least longitudinal offset distance 404 and to cardiac tissue surrounding first electrically active region 216.

For some locations for target implantation region 106, the surface of atrial endocardium 604 may be curved, may include protrusions, and/or may otherwise not define a substantially flat surface. The features on the surface of atrial endocardium 604 may require implantation of device 104 into the surface at an angle (e.g., where longitudinal axis 210 of device 104 is not orthogonal to the surface of atrial endocardium 604. In such examples, second electrode 114 may at least partially compress during implantation, which may increase fixation of device 104 to atrial endocardium 604 and increase the contact between second electrically active region 217 and the surface of atrial endocardium 604.

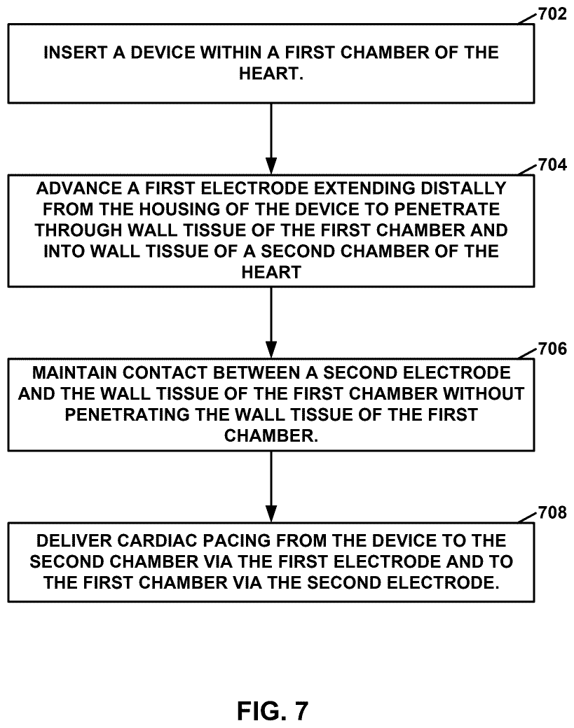

FIG. 7 is a flowchart illustrating an example process for sensing a cardiac electrical signal and delivering cardiac pacing therapy to a heart of a patient via an example device of any of FIGS. 1-6. The technique of FIG. 7 will be described with concurrent reference to device 104 as illustrated in FIGS. 1-6, although a person having ordinary skill in the art will understand that the technique may be performed in reference to an implantable medical lead or other medical device.

A clinician may insert device 104 within a single first chamber of the heart 102 (702). The first chamber of heart 102 may be the right atrium, left atrium, the right ventricle, or the left ventricle. The clinician may insert device 104 into the first chamber via delivery tool connected to device 104 (e.g., connected to delivery tool interface member 208). The clinician may advance first electrode 112 extending distally from housing 202 of device 104 to penetrate through wall tissue of the first chamber and into wall tissue of a second chamber of heart 102 (704). For example, the clinician may advance distal tip 302 of distal end 220 of first electrode 112 to a distance 608 within the wall tissue. In some examples, advancing first electrode 112 includes positioning distal end 220 of first electrode 112 within ventricular myocardium 108 of the patient and placing first electrically active region 216 of first electrode 112 proximal to distal end 220 (e.g., at least longitudinal offset distance 404 from distal tip 302 of first electrode 112). The clinician may advance first electrode 112 by rotating device 104 clockwise or counterclockwise within the first chamber, depending on how first electrode 112 is wound. Depending on the surface features (e.g., surface curvature) of tissue within the target implantation region 106, the clinician may implant device 104 into the tissue orthogonally or at an angle. In some examples when device 104 is implanted into tissue at an angle, distance 608, longitudinal offset distance 404, and longitudinal length 402 of first electrode 112 may be measured along longitudinal axis 210 of device 104.

The clinician may cause device 104 to maintain contact between second electrode 114 and the wall tissue of the first chamber, without penetrating the wall tissue of the first chamber (706). The clinician may continue to advance first electrode 112 into the tissue until at least a portion of face 205 of distal end 204 of device 104 contacts the surface of the tissue. Once device 104 is implanted within the wall tissue, second electrode 114 and/or one or more fixation features may interface with the wall tissue to inhibit unintended movement of first electrode 112 out of the wall tissue.

Device 104 may deliver cardiac pacing from device 104 to the second chamber via first electrode 112 and to the first chamber via second electrode 114 (708). Device 104 may deliver cardiac pacing to the first chamber and/or the second chamber via first electrode 112, second electrode 114, and/or one or more other electrodes of device 104 (e.g., electrode 218). Device 104 may deliver cardiac pacing to the second chamber via first electrically active region 216 of first electrode 112, e.g., proximal to distal end 220 of first electrode 112. Device 104 may deliver cardiac pacing at least longitudinally offset distance 404 from distal tip 302 of first electrode 112, e.g., to reduce pacing capture thresholds of cardiac tissue of the second chamber, to reduce the effects of the lateral movement of first electrode 112 on the effects of cardiac pacing, or to reduce delivery of cardiac pacing to inflamed and/or irritated cardiac tissue.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

In addition, it should be noted that system described herein may not be limited to treatment of a human patient. In alternative examples, the system may be implemented in non-human patients, e.g., primates, canines, equines, pigs, and felines. These other animals may undergo clinical or research therapies that may benefit from the subject matter of this disclosure.

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

This disclosure describes each of the following examples.

Example 1: a device comprising: an elongated housing extending from a proximal end to a distal end along a longitudinal axis, the elongated housing being configured to be implanted wholly within a chamber of a heart; an elongated body extending distally from the distal end of the elongated housing, the elongated body comprising: a proximal portion extending distally from the distal end of the elongated housing, a medial portion coupled to and distal to the proximal portion, the medial portion defining a first electrode, and a distal portion coupled to and distal to the medial portion, the distal portion defining a distal end of the elongated body, wherein the distal portion is configured to penetrate wall tissue of the chamber; and a second electrode disposed on the distal end of the elongated housing, the second electrode being configured to interface with the wall tissue of the chamber without penetrating the wall tissue.

Example 2: the device of example 1, wherein the elongated body defines a helix.

Example 3: the device of any of examples 1 or 2, wherein the medial portion extends from a first end to a second end, wherein the first end of the medial portion is connected to a distal end of the proximal portion, wherein the second end of the medial portion is connected to a proximal end of the distal portion.

Example 4: the device of example 3, wherein the second end of the medial portion is separated from the distal end of the elongated body along the longitudinal axis by a distance of up to 2 millimeters (mm).

Example 5: the device of any of examples 1-4, wherein the medial portion defines a surface area of up to 5 square millimeters (mm²).

Example 6: the device of example 5, wherein the medial portion defines a surface area of up to 3.5 mm².

Example 7: the device of any of examples 1-6, wherein the proximal portion is formed from a first material, wherein the medial portion comprises a second material, wherein the distal portion comprises a third material, and wherein the second material is different from one or more of the first material or the third material.

Example 8: the device of example 7, wherein the first material and the third material comprise one or more electrically insulating materials, and wherein the second material comprises one or more electrically conductive materials.

Example 9: the device of any of examples 1-8, wherein the elongated body comprises one or more electrically insulating materials disposed over one or more of the proximal portion or the distal portion.

Example 10: the device of example 9, wherein the one or more electrically insulating materials comprises one or more of silicon or polytetrafluoroethylene (PTFE).

Example 11: the device of any of examples 1-10, wherein the first electrode is configured to deliver a cardiac pacing signal to cardiac tissue of the heart at or around a region proximal to the distal end of the elongated body along the longitudinal axis.

Example 12: the device of any of examples 1-11, wherein the chamber of the heart comprises a first chamber of the heart, and wherein the elongated body is configured to penetrate into wall tissue of a second chamber of the heart that is separated from the first chamber of the heart.

Example 13: the device of example 12, wherein the first chamber comprises an atrium of the heart, and wherein the second chamber comprises a ventricle of the heart.

Example 14: a method comprising: penetrating, via an elongated body of an implantable medical device (IMD), tissue within a first chamber of a heart of a patient, wherein the elongated body comprises: a proximal portion extending distally from a distal end of an elongated housing of the IMD along a longitudinal axis of the IMD, a medial portion coupled to and distal to the proximal portion, the medial portion defining a first electrode, and a distal portion coupled to and distal to the medial portion, the distal portion defining a distal end of the elongated body; advancing the elongated body of the IMD into the tissue of the heart until a second electrode of the IMD contacts the wall tissue without penetrating the tissue, wherein when the second electrode contacts the tissue, the distal end of the elongated body is positioned at a first location within the tissue of the heart; delivering, via the first electrode of the IMD, cardiac pacing to a second chamber of the heart, wherein the second chamber is separate from the first chamber, wherein delivering the cardiac pacing to the second chamber comprises: delivering, via the first electrode, the cardiac pacing to a second location within the tissue of the heart, the second location being proximal to the first location along the longitudinal axis; and delivering, via the second electrode of the IMD, cardiac pacing to the first chamber of the heart.

Example 15: the method of example 14, wherein the elongated body of the IMD defines a helix.

Example 16: the method of any of examples 14 or 15, wherein the medial portion extends from a first end to a second end, wherein the first end of the medial portion is connected to a distal end of the proximal portion, and wherein the second end of the medial portion is connected to a proximal end of the distal portion.

Example 17: the method of example 16, wherein the second end of the medial portion is separated from the distal end of the elongated body along the longitudinal axis by a distance of up to 2 millimeters (mm).

Example 18: the method of any of examples 14-17, wherein the medial portion defines a surface area of up to 5 square millimeters (mm²).

Example 19: the method of example 18, wherein the medial portion defines a surface area of up to 3.5 mm².

Example 20: the method of any of examples 14-19, wherein the proximal portion is formed from a first material, wherein the medial portion comprises a second material, wherein the distal portion comprises a third material, and wherein the second material is different from one or more of the first material or the third material.

Example 21: the method of example 20, wherein the first material and the third material comprise one or more electrically insulating materials, and wherein the second material comprises one or more electrically conductive materials.

Example 22: the method of any of examples 14-21, wherein the elongated body comprises one or more electrically insulating materials disposed over one or more of the proximal portion or the distal portion.

Example 23: the method of example 22, wherein the one or more electrically insulating materials comprises one or more of silicon or polytetrafluoroethylene (PTFE).

Example 24: the method of any of examples 14-23, wherein the first chamber comprises an atrium of the heart, and wherein the second chamber comprises a ventricle of the heart.

Example 25: the method of any of examples 14-24, wherein the second electrode is disposed on the distal end of the elongated housing of the IMD.

Example 26: the method of any of examples 14-25, wherein the second location is adjacent to cardiac tissue of the second chamber.

Example 27: a method comprising: disposing, by a manufacturing process, an electrically insulating material over an outer surface of an elongated body, the elongated body comprising: a proximal portion; a medial portion coupled to and distal to the proximal portion; and a distal portion coupled to and distal to the medial portion, the distal portion defining a distal end of the elongated body; removing, by the manufacturing process, a portion of the electrically insulating material around the medial portion of the elongated body to expose an outer surface of the medial portion; and either before or after said disposing, assembling, by the manufacturing process, the elongated body to an implantable medical device (IMD), the IMD comprising: an elongated housing extending from a proximal end to a distal end along a longitudinal axis, the elongated housing being configured to be implanted wholly within a chamber of a heart.

Example 28: the method of example 27, wherein the elongated body defines a helix.

Example 29: the method of any of examples 27 or 28, wherein removing the portion of the electrically insulating material around the medial portion of the elongated body comprises: removing, by the manufacturing process, the portion of the electrically insulating material around the medial portion of the elongated body via laser ablation.

Example 30: the method of any of examples 27-29, wherein removing the portion of the electrically insulating material around the medial portion of the elongated body comprises: removing, by the manufacturing process, the portion of the electrically insulating material around the medial portion of the elongated body via a cutting instrument.

Example 31: the method of any of examples 27-30, further comprising: forming, by the manufacturing process, an electrically conductive material into the elongated body, wherein the elongated body defines an electrically conductive outer surface.

Example 32: the method of any of examples 27-31, wherein the one or more electrically insulating materials comprises polytetrafluoroethylene (PTFE).

Example 33: the method of any of examples 27-32, wherein the outer surface of the medial portion defines an electrode of the IMD.

Example 34: a medical device comprising: a housing extending from a proximal end to a distal end along a longitudinal axis, the housing being configured to be implanted wholly within a chamber of a heart; a helix extending in a distal winding direction from the distal end of the elongated housing, the helix comprising a distal portion configured to penetrate wall tissue of the chamber; a first electrode formed or carried by the helix, the first electrode being located at a position on the helix that is spaced in a proximal winding direction from the distal portion of the helix.

Example 35: the medical device of example 34, further comprising a second electrode at the distal end of the elongated housing, the second electrode being configured to interface with the wall tissue of the chamber without penetrating the wall tissue.

Example 36: the medical device of any of examples 34 or 35, wherein the first electrode comprises a proximal end and a distal end, the distal end being spaced in the distal winding direction from the proximal end, wherein the distal end of the first electrode is spaced in the proximal winding direction from a distal tip of the helix.

Example 37: the medical device of any of examples 34-36, wherein the first electrode is spaced from the distal portion of the helix by a distance of up to 2 millimeters (mm).

Example 38: the medical device of any of examples 34-36, wherein the first electrode defines a surface area of up to 5 square millimeters (mm²).

Example 39: the medical device of example 38, wherein the first electrode defines a surface area of up to 3.5 mm².

Example 40: the medical device of any of examples 34-39, wherein the distal portion is formed from a first material, wherein the first electrode is formed from a second material, and wherein the first material is different from the second material.

Example 41: the medical device of example 40, wherein the first material comprises one or more electrically insulating materials, and wherein the second material comprises one or more electrically conductive materials.

Example 42: the medical device of any of examples 34-41, wherein the first electrode is configured to deliver a cardiac pacing signal to cardiac tissue of the heart at or around a region proximal in the proximal winding direction to a distal tip of the helix.

Various examples have been described. These and other examples are within the scope of the following claims.

What is claimed is:

1. A device comprising:
an elongated housing extending from a proximal end to a distal end along a longitudinal axis, the elongated housing being configured to be implanted wholly within a chamber of a heart;
an elongated body extending distally from the distal end of the elongated housing, the elongated body comprising:
a proximal portion extending distally from the distal end of the elongated housing,
a medial portion coupled to and distal to the proximal portion, the medial portion defining a first electrode, and
a distal portion coupled to and distal to the medial portion, the distal portion defining a distal end of the elongated body, wherein the distal portion is configured to penetrate wall tissue of the chamber; and
a second electrode disposed on the distal end of the elongated housing, the second electrode being configured to interface with the wall tissue of the chamber without penetrating the wall tissue.

2. The device of claim 1, wherein the elongated body defines a helix.

3. The device of claim 1, wherein the medial portion extends from a first end to a second end, wherein the first end of the medial portion is connected to a distal end of the proximal portion, wherein the second end of the medial portion is connected to a proximal end of the distal portion.

4. The device of claim 3, wherein the second end of the medial portion is separated from the distal end of the elongated body along the longitudinal axis by a distance of up to 2 millimeters (mm).

5. The device of claim 1, wherein the medial portion defines a surface area of up to 5 square millimeters (mm²).

6. The device of claim 1, wherein the proximal portion is formed from a first material, wherein the medial portion comprises a second material, wherein the distal portion comprises a third material, and wherein the second material is different from one or more of the first material or the third material.

7. The device of claim 6, wherein the first material and the third material comprise one or more electrically insulating materials, and wherein the second material comprises one or more electrically conductive materials.

8. The device of claim 1, wherein the elongated body comprises one or more electrically insulating materials disposed over one or more of the proximal portion or the distal portion.

9. The device of claim 1, wherein the first electrode is configured to deliver a cardiac pacing signal to cardiac tissue of the heart at or around a region proximal to the distal end of the elongated body along the longitudinal axis.

10. The device of claim 1, wherein the chamber of the heart comprises a first chamber of the heart, and wherein the elongated body is configured to penetrate into wall tissue of a second chamber of the heart that is separated from the first chamber of the heart.

11. A method comprising:
penetrating, via an elongated body of an implantable medical device (IMD), tissue within a first chamber of a heart of a patient, wherein the elongated body comprises:
a proximal portion extending distally from a distal end of an elongated housing of the IMD along a longitudinal axis of the IMD,
a medial portion coupled to and distal to the proximal portion, the medial portion defining a first electrode, and
a distal portion coupled to and distal to the medial portion, the distal portion defining a distal end of the elongated body;
advancing the elongated body of the IMD into the tissue of the heart until a second electrode of the IMD contacts the wall tissue without penetrating the tissue, wherein when the second electrode contacts the tissue, the distal end of the elongated body is positioned at a first location within the tissue of the heart;
delivering, via the first electrode of the IMD, cardiac pacing to a second chamber of the heart, wherein the second chamber is separate from the first chamber, wherein delivering the cardiac pacing to the second chamber comprises:
delivering, via the first electrode, the cardiac pacing to a second location within the tissue of the heart, the second location being proximal to the first location along the longitudinal axis; and
delivering, via the second electrode of the IMD, cardiac pacing to the first chamber of the heart.

12. The method of claim 11, wherein the medial portion extends from a first end to a second end, wherein the first end of the medial portion is connected to a distal end of the proximal portion, and wherein the second end of the medial portion is connected to a proximal end of the distal portion.

13. A medical device comprising:
a housing extending from a proximal end to a distal end along a longitudinal axis, the housing being configured to be implanted wholly within a chamber of a heart;
a helix extending in a distal winding direction from the distal end of the elongated housing, the helix comprising a distal portion configured to penetrate wall tissue of the chamber;
a first electrode formed or carried by the helix, the first electrode being located at a position on the helix that is spaced in a proximal winding direction from the distal portion of the helix.

14. The medical device of claim 13, wherein the first electrode comprises a proximal end and a distal end, the distal end being spaced in the distal winding direction from the proximal end, wherein the distal end of the first electrode is spaced in the proximal winding direction from a distal tip of the helix.

15. The medical device of claim 13, wherein the first electrode is configured to deliver a cardiac pacing signal to cardiac tissue of the heart at or around a region proximal in the proximal winding direction to a distal tip of the helix.

16. The medical device of claim 13, further comprising a second electrode at the distal end of the elongated housing, the second electrode being configured to interface with the wall tissue of the chamber without penetrating the wall tissue.

17. The medical device of claim 13, wherein the first electrode is spaced from the distal portion of the helix by a distance of up to 2 millimeters (mm).

18. The medical device of claim 13, wherein the first electrode defines a surface area of up to 5 square millimeters (mm²).

19. The medical device of claim 13, wherein the distal portion is formed from a first material, wherein the first electrode is formed from a second material, and wherein the first material is different from the second material.

20. The medical device of claim 19, wherein the first material comprises one or more electrically insulating materials, and wherein the second material comprises one or more electrically conductive materials.
